(19) United States
(12) Patent Application Publication
(10) Pub. No.: US 2016/0325274 A1
(43) Pub. Date: Nov. 10, 2016

(54) CATALYST COMPOSITIONS FOR ETHYLENE DIMERIZATION

(71) Applicant: SAUDI BASIC INDUSTRIES CORPORATION, Riyadh (SA)

(72) Inventor: Roland Schmidt, Wiehl (DE)

(73) Assignee: Saudi Basic Industries Corporation, Riyadh (SA)

(21) Appl. No.: 15/109,732

(22) PCT Filed: Dec. 22, 2014
(86) PCT No.: PCT/IB14/67244

§ 371 Date: Jul. 6, 2016

Related U.S. Application Data
(60) Provisional application No. 61/923,961, filed on Jan. 6, 2014.

(51) Int. Cl. B01J 31/22; C08F 10/08; C07C 2/32; B01J 31/14; B01J 31/02

(52) U.S. Cl. CPC B01J 31/2208; B01J 31/143; B01J 31/0204; C07C 2/32; C08F 10/08; B01J 2231/20; B01J 2531/46; C07C 2531/14; C07C 2531/22

(57) ABSTRACT

Catalyst compositions that are suitable for producing 1-butene are provided. In an exemplary embodiment, the catalyst compositions include an organic titanium compound, an organic aluminum compound, and a linear diether. Processes for converting ethylene to 1-butene by using these catalyst compositions are also provided.

### FIELD

The presently disclosed subject matter relates to catalyst compositions that are suitable for catalytic dimerization of ethylene to produce 1-butene and processes for converting ethylene to 1-butene.

### BACKGROUND

The compound 1-butene has for a long time been a desirable substance in the chemical industry. Not only can 1-butene be converted to polybutene-1 and butylene oxides, it can also be used as a co-monomer with ethylene for the production of high strength and high stress crack resistant polyethylene resins. The major industrial routes for producing 1-butene include steam cracking of C₄ hydrocarbon streams, ethylene oligomerization processes, refinery operations of crude oil, and ethylene dimerization processes. Catalytic dimerization of ethylene into 1-butene produces higher chain polymers via the growth reaction of the organoaluminum compounds (Ziegler, Angew. Chem. (1952); 64:323-329; J. Boor, Editor, Ziegler-Natta Catalysts and Polymerizations, Acad. Press (New York) 1979; Handbook of Transition Metal Polymerization Catalysts, R. Hoff, R. T. Mathers, Eds. 2010 John Wiley & Sons).

One route to the preparation of 1-butene is the cracking of higher petrochemical fractions containing more than four carbon atoms. A further route to the preparation of 1-butene is via the catalytic dimerization of ethylene. The industrial synthesis of 1-butene can be achieved using nickel or titanium catalysts in large industrial processes such as Alphabutol™ (Handbook of Petroleum Processing, Edited by D. S. J. Jones, P. R. Pujadó; Springer Science 2008; Forestiere et al., Oil & Gas Science and Technology-Rev. IFP (2009); 64(6):649-667). The catalytic activity of the Alphabutol™ system can be low, at roughly 1 kg of product per gram of titanium. Polymer formation and lengthy initial induction period are major drawbacks for the commercial Alphabutol™ system.

There remains a need in the art for a catalyst composition that is suitable for dimerization of ethylene, which catalyst composition has one or more of improved catalytic activity, shortened induction period, reduced polymer formation, long lifetimes, and high selectivity.

### SUMMARY

The presently disclosed subject matter provides catalyst compositions that are suitable for catalytic dimerization of ethylene, e.g., to produce 1-butene, and processes for converting ethylene to 1-butene. In some embodiments, an example catalyst composition includes an organic titanium compound, an organic aluminum compound, and a linear diether. The organic titanium compound can be titanium tetra-n-butoxide. The organic aluminum compound can be triethylaluminum. The linear diether can be dimethoxyethane, diethoxyethane, or combinations thereof. The dimethoxyethane can be 1,2-dimethoxyethane. The catalyst composition can further include tetrahydrofuran. The catalyst compositions are suitable for converting ethylene to 1-butene.

The presently disclosed subject matter also provides processes for converting ethylene to 1-butene. In some embodiments, an example process includes contacting ethylene with a catalyst composition, which includes an organic titanium compound, an organic aluminum compound, and a linear diether. The organic titanium compound can be titanium tetra-n-butoxide. The organic aluminum compound can be triethylaluminum. The linear diether can be dimethoxyethane, diethoxyethane, or combinations thereof. The dimethoxyethane can be 1,2-dimethoxyethane. In certain embodiments, the total ethylene consumption after about 1 hour is about 125 g when the process is performed at a temperature of about 55° C. to about 60° C. and a pressure of from about 20 Bars to about 25 Bars. The catalyst composition can further include tetrahydrofuran.

### BRIEF DESCRIPTION OF THE DRAWINGS

FIG. 1 represents the total ethylene consumptions of various catalyst compositions.

FIG. 2 illustrates a catalytic dimerization of ethylene process to produce 1-butene in the presence of an organic titanium compound being a main catalyst.

FIG. 3 illustrates the ethylene consumption of a catalyst composition including TNBT, THF, and TEAL.

### DETAILED DESCRIPTION

The presently disclosed subject matter provides catalyst compositions that are suitable for producing 1-butene and processes for converting ethylene to 1-butene.

The catalyst compositions of the presently disclosed subject matter include (1) an organic titanium compound, (2) an organic aluminum compound, and (3) a diether. In the catalyst composition, the organic titanium compound can be the main catalyst in the catalyst composition. The organic aluminum compound can be a co-catalyst, which can release free coordination sites in the titanate complex, can withdraw electron density from around the titanium metal center, and can generate one or more Ti—C bonds by exchanging its ethyl groups with the butoxide groups of titanate complex.

The organic titanium compound can be an alkyl titanate having a general formula of Ti(OR)₄, where R is a linear or branched alkyl radical having from about 1 to about 12 carbon atoms, i.e., a C₂-C₁₂ alkyl group, a C₂-C₈ alkyl group, or a C₃-C₅ alkyl group. In some embodiments, the alkyl group is butyl, preferably n-butyl. Suitable organic titanium compounds include, but are not limited to, tetraethyl titanate, tetraisopropyl titanate, titanium tetra-n-butoxide (TNBT), and tetra-2-ethyl-hexyl titanate. In some embodiments, the organic titanium compound is titanium tetra-n-butoxide.

The organic titanium compound can be present in high concentration in the catalyst composition. In some embodiments, the organic titanium compound is present in a concentration of from about 0.0001 to about 0.1 mol/dm³, from about 0.0001 to about 0.0005 mol/dm³, from about 0.0005 to about 0.001 mol/dm³, from about 0.001 to about 0.01 mol/dm³, from about 0.01 to about 0.1 mol/dm³.

The organic aluminum compound can have the general formula Al(R)₃, where R can be a hydrocarbon, for example a C₁-C₁₂ hydrocarbon, H or a halogen. Each R in a molecule may be the same as or different to the other R groups in the molecule. Organic aluminum compounds are known to one of ordinary skill in the art and the artisan can select the organic aluminum compounds in order to enhance the advantageous properties of the process according to the presently disclosed subject matter. In some embodiments, R is an alkyl group. R can be a straight chain or branched chain alkyl group. In some embodiments, R is a straight chain alkyl group. R can be a C₁-C₁₂ alkyl group, a C₁-C₈ alkyl group, or a C₁-C₄ alkyl group. In some embodiments, the alkyl group is ethyl. Suitable organic aluminum compounds include, but are not limited to, triethylaluminum (TEAL), trimethylaluminum (TMA), tri-n-propylaluminum, triisobutylaluminum, diisobutylaluminum hydride, and trihexylaluminum. In some embodiments, the organic aluminum compound is aluminum trialkyls, which can be triethylaluminum and trimethylaluminum.

The diether of the presently disclosed subject matter is a modifier. Modifiers are polar additives that when added to the catalyst composition and can effect changes in the nature of active centers and have a profound effect on the catalytic activity and selectivity. Without being bound by theory, modifiers can stabilize the titanium (IV) complex and prevent from the formation titanium (III) complex which is responsible for the production of heavy compounds. The diether of the presently disclosed subject matter can be a linear diether or a cyclic diether.

In some embodiments, an example diether is a cyclic diether, in particular a substituted or unsubstituted cyclic diether having a total of 3 to 14 carbon atoms, or 4 to 10 carbon atoms, or 4 to 8 carbon atoms. Cyclic diethers can be dioxanes, including, but not limited to, 1,4-dioxane, and alkyl, aryl, alkenyl, or halogenated substituted derivatives thereof and combinations thereof, for example 1,4-dioxane optionally substituted with 1, 2, or 3 halogen, C₁-C₆ alkyl, C₆₋₁₀ aryl, or C₂-C₆ alkenyl substituents, or combinations thereof, preferably 1 or 2 substituents selected from halogen or C₁-C₃ alkyl or combinations thereof. In some embodiments, the cyclic diether is 1,4-dioxane.

In another embodiment, one example diether is a linear diether. Suitable linear diethers include, but are not limited to dimethoxyethane, diethoxyethane, and symmetrically or unsymmetrically alkyl, aryl, alkenyl, or halogenated substituted diethers, and combinations thereof. The dimethoxyethane can be 1,2-dimethoxyethane (1,2-DME). For example, in some embodiments the linear diether is of the formula A-O—B—O—C wherein A, B, and C are the same or different, and are a C₁-C₆ alkyl, C₆₋₁₀ aryl, or C₂-C₆ alkenyl, each of which can be optionally substituted with one or more halogens. In some embodiments, A, B, and C are the same or different, and are a C₁-C₃ alkyl, phenyl, or C₂-C₄ alkenyl, each of which can be optionally substituted with one or more halogen. The linear diether can be a symmetrical or unsymmetrical diether of the formula (CₙH₂ₙ₊₁)O(CₙH₂ₙ)O(CₙH₂ₙ₊₁) wherein for each moiety n is the same or different, and is 1 to 4, or is 1 to 2.

The commercial Alphabutol™ system is a homogeneous catalyst composition for selective ethylene dimerization reaction. The commercial Alphabutol™ system includes TNBT as the main catalyst and TEAL as a co-catalyst, and TNBT is mixed with tetrahydrofuran (THF) at a volume ratio of about 1. The commercial Alphabutol™ system is associated with low catalytic activity, a lengthy induction period, and process fouling (precipitation of polyethylene). Completely or partially substituting THF with the presently disclosed diether can increase the catalytic activity and/or selectivity of the system, reduce or prevent fouling (reduce polymer (polyethylene) formation), and shorten the induction period. The catalyst composition of the presently disclosed subject matter can increase the catalytic activity of the system, e.g., when the system is used for converting ethylene to 1-butene.

The catalytic activity of the presently disclosed catalyst composition can be at least about 40% greater, at least about 50% greater, at least about 60% greater, at least about 70% greater, at least about 80% greater, at least about 90% greater, at least about 100% greater, at least about 105% greater, at least about 108% greater, at least about 110% greater, at least about 120% greater, at least about 130% greater, at least about 140% greater, or at least about 150% greater than the catalytic activity of the commercial Alphabutol™ system.

In some embodiments, the catalytic activity of the presently disclosed catalyst composition is about 108% greater than the catalytic activity of the commercial Alphabutol™ system. In another embodiment, the catalytic activity of the presently disclosed catalyst composition is about 50% greater than the catalytic activity of the commercial Alphabutol™ system. The catalytic activity can be measured or evaluated based on the ethylene consumption in the process of converting ethylene to 1-butene by using a catalyst composition, e.g., the presently disclosed catalyst composition and the commercial Alphabutol™ system. In some embodiments, the diether is 1,2-dimethoxyethane (1,2-DME), and the ethylene consumption after about 1 hour at a temperature of from about 55° C. to about 60° C. and a pressure of from about 20 Bars to about 25 Bars is about 125 g, which is about 108% greater than the ethylene consumption of the commercial Alphabutol™ system under the same reaction conditions. In some embodiments, the diether is 1,4-dioxane, and the ethylene consumption after about 1 hour at a temperature of from about 55° C. to about 60° C. and a pressure of from about 20 Bars to about 25 Bars is about 86 g, which is about 50% greater than the ethylene consumption of the commercial Alphabutol™ system under the same reaction conditions.

The amount (e.g., molar %, volume %, or weight %) of the diether included in the catalyst composition can impact the catalytic activity of the system. In addition, when the catalyst composition is used for producing 1-butene via catalytic dimerization of ethylene, the amount (e.g., molar %, volume %, or weight %) of the diether can impact the polymer (e.g., polyethylene) formation during the production process. In certain embodiments, the diether is a cyclic diether, in particular 1,4-dioxane, which is present in the catalyst composition in a volume % of from about 1% to about 99%, from about 10% to about 90%, from about 20% to about 80%, from about 30% to about 70%, from about 40% to about 70%, or from about 50% to about 60%. In certain embodiments, the catalyst composition includes from about 30 vol % to about 60 vol % of the cyclic diether. In certain embodiments, the diether is a linear diether, in particular 1,2-DME, which is present in the catalyst composition in a volume % of from about 1% to about 99%, from about 10% to about 90%, from about 20% to about 80%, from about 30% to about 70%, from about 40% to about 70%, or from about 50% to about 60%. In certain embodiments, the catalyst composition includes from about 30 vol % to about 60 vol % of the linear diether.

The catalyst composition can further include a second solvent to mix with the organic titanium compound, in particular a monoether. The second solvent can be THF. In certain embodiments, the combination of a second solvent with a diether can provide an unexpected and surprising synergistic effect on the catalytic activity of the system. For example, the catalytic activity of the catalyst composition (more particularly, of the main catalyst, which is the organic titanium compound) including a combination of a second solvent and a diether can be unexpectedly and surprisingly higher than the additive catalytic activity of a catalyst composition including only a second solvent plus the catalytic activity of a catalyst composition including only a diether. The volume ratio of the diether to the monoether can be 1:99 to 99:1, for example 80:20 to 20:80.

The catalyst compositions of the presently disclosed subject matter can be used for catalytic dimerization of ethylene, e.g., to produce an α-olefin (e.g., 1-butene). Catalytic dimerization of ethylene can be carried out as a flow reaction or a batch reaction. Catalytic dimerization of ethylene can proceed as a homogeneous reaction (e.g., in the liquid phase), or as a heterogeneous reaction. In some embodiments, catalytic dimerization of ethylene proceeds as a homogeneous liquid phase reaction.

The organic titanium compound and organic aluminum compound of the can be employed in the form of diluted solutions. Suitable solvents include, but are not limited to, aliphatic hydrocarbons (e.g., butene, pentane, hexane, heptane), aromatic hydrocarbons (benzene, toluene), olefins (1-butene, pentenes, hexenes), or combinations thereof.

The catalyst composition can be pre-prepared, wherein the components of the composition are dissolved in a liquid, preferably to form a homogenous composition. The liquid can be, for example, a C₄-C₁₂ alkane, a C₄-C₈ alkane, or a C₄-C₆ alkane such as hexane; or a C₄-C₁₂ alkene, for example a C₄-C₈ alkene, or a C₄-C₆ alkene such as butene. The liquid can be one or more selected from butene, hexane, heptane, and octane. Alternatively, the catalyst composition can be prepared in situ, that is, the catalyst composition can be introduced to the reaction system in at least two or more components, which are added sequentially. For example, the organic aluminium compound diluted in an inhibitor (e.g., THF) and the organic titanium compound diluted in a solvent (e.g., n-hexane) can be added to the reactor sequentially.

In some embodiments, the catalyst composition is prepared shortly before use. For example, the prepared catalyst composition is not stored for longer than 1 week, not longer than 1 day, or not longer than 5 hours before being employed for catalytic dimerization of ethylene.

In some embodiments, the organic titanium compound is not activated until shortly before being employed for catalytic dimerization of ethylene. For example, the organic aluminium compound diluted in an inhibitor is not brought into contact with the organic titanium compound to activate the latter earlier than 30 minutes, not earlier than 15 minutes, not earlier than 10 minutes, not earlier than 5 minutes before the catalyst composition is employed for catalytic dimerization of ethylene.

In some embodiments, the individual components are prepared shortly before use. For example, at least one or more of the catalyst components are not stored for longer than 1 week, not longer than 1 day, not longer than 5 hours after its preparation and before being employed as a constituent of the catalyst composition for catalytic dimerization of ethylene. In one aspect of this embodiment, the organic titanium compound is not stored for longer than 1 week, not longer than 1 day, not longer than 5 hours after its preparation and before being employed as a constituent of the catalyst composition for catalytic dimerization of ethylene. In one aspect of this embodiment, the organic aluminium compound diluted in an inhibitor is not stored for longer than 1 week, not longer than 1 day, not longer than 5 hours after its preparation and before being employed as a constituent of the catalyst composition for catalytic dimerization of ethylene.

Catalytic dimerization of ethylene can be performed at a temperature of from about 20° C. to about 150° C., from about 40° C. to about 100° C., from about 20° C. to about 70° C., from about 50° C. to about 70° C., from about 50° C. to about 55° C., or from about 55° C. to about 65° C. In some embodiments, catalytic dimerization of ethylene is performed at a temperature of about 60° C.

Catalytic dimerization of ethylene can be performed at a pressure of from about 5 bars to about 50 bars, from about 10 bars to about 40 bars, or from about 15 bars to about 30 bars. Catalytic dimerization of ethylene can be conducted in a batch, and a selected volume of the presently disclosed catalyst composition can be introduced into a reactor provided with usual stirring and cooling systems, and can be subjected therein to an ethylene pressure, which can be from about 22 bars to about 27 bars. In some embodiments, catalytic dimerization of ethylene using the presently disclosed catalyst composition is conducted at an ethylene pressure of about 23 bar. One of ordinary skill in the art can adjust the temperature, pressure and other conditions of the reaction in order to bring about favorable properties of the reaction, for example, in order to ensure that the reaction system is present as a homogeneous liquid phase. The reaction product (e.g., 1-butene) can be extracted by any methods which one of ordinary skill in the art would consider to suitable in the context of the presently disclosed subject matter. Suitable methods of extraction include, but are not limited to, distillation, precipitation, crystallization, and membrane permeation.

The process for catalytic dimerization of ethylene to produce 1-butene can be coupled to further subsequent reactions in order to obtain downstream products. Downstream products are those obtained from polymerisation reactions, hydrogenation reactions, halogenation reactions, and other chemical functionalization reactions. The chemical functionalization products can be aromatic or non-aromatic compounds, saturated or unsaturated compounds, ketones, aldehydes, esters, amides, amines, carboxylic acids, alcohols etc. Monomeric downstream products can be chloro-butene, butadiene, butanol, or butanone. In certain embodiments, the downstream products are those obtained from polymerisation reactions. Polymerization reactions can be mono-polymerization reactions or co-polymerization reactions. The polymerisation product can be poly-butene. Co-polymers can include α-olefin (e.g., 1-butene) and one or more co-monomers including, but not limited to: ethene, propene, pentene, styrene, acrylic acid, methacrylic acid, methyl acrylate, methyl methacrylate, acrylonitrile, methacrylonitrile, or vinyl chloride. In certain embodiments, the co-polymer is a co-polymer of ethylene and 1-butene. The ethylene monomers can be present in a greater wt. % the than the 1-butene monomers in the co-polymer. For example, the weight ratio of ethylene monomers to 1-butene monomers can be from about 50:1 to about 5:1, from about 30:1 to about 10:1, or from about 25:1 to about 15:1. One or ordinary skill in the art can vary the ratio relating the mass of ethylene monomers and 1-butene monomers in order to tune the desired properties of polythene or polypropene, such as crystallinity and elasticity.

In some embodiments of the process for preparing a downstream product, the product includes compounds with chain lengths in proportions determined by or approximating to the Anderson Schulz Flory distribution (see P. L. Spath and D. C. Dayton. “Preliminary Screening Technical and Economic Assessment of Synthesis Gas to Fuels and Chemicals with Emphasis on the Potential for Biomass-Derived Syngas”, NREL/TP510-34929, December, 2003, pp. 95).

In some embodiments, the downstream products are further connected to yield fatty acids, e.g., with chain lengths in proportions determined by or approximating to the Anderson Schulz Flory distribution.

In some embodiments, the downstream products are further processed, particularly in the case where the downstream product is a polymer, particularly when it is a polybutene homopolymer or copolymer. In one aspect of this embodiment, this further processing can involve formation of shaped objects such as plastic parts for electronic devices, automobile parts, such as bumpers, dashboards, or other body parts, furniture, or other parts or merchandise, or for packaging, such as plastic bags, film, or containers.

### EXAMPLES

The following examples are merely illustrative of the presently disclosed subject matter and they should not be considered as limiting the scope of the presently disclosed subject matter in any way.

The following experiments were conducted in a 300 ml jacketed Parr autoclave reactor (Parr Modell 4566). The standard conditions were 23 bars ethylene pressure and 60° C. for 1 hour.

### Example 1

2.75 ml of TNBT was mixed with 2.65 ml of 1,4-dioxane (with a density p of 1.03). About 2.2 ml of 1M TEAL dissolved in 50 ml of n-hexane was added to 50 ml of the mixture of TNBT/1,4-dioxane to obtain a catalytic solution. The catalytic solution was added to the reactor by vacuum suction. The reactor was then pressurized with ethylene from a 2-liter aluminum gas cylinder (ethylene supply) to reach the desired pressure (23 bars in most experiments). The reaction pressure was controlled using backpressure regulator, while the ethylene consumption was measured using a balance onto which the gas cylinder was placed. The reactor was equipped with a thermocouple to measure the temperature inside the reactor. Temperature, pressure, and ethylene consumption data were recorded using a data acquisition system. Prior to the catalytic solution injection, the reactor heated to 80° C. under vacuum for at least two hours under vacuum in order to eliminate all traces of moisture. The temperature was controlled by a heating mantle/furnace and cooling coil refrigeration. After terminating the reaction by depressurization, the product was collected, hydrolyzed with deionized water, and analyzed by GC and/or GC/MS. Total ethylene consumption was measured by weight difference of the attached ethylene storage cylinder.

The commercial Alphabutol™ system catalyst batch tested for ethylene dimerization to 1-butene was evaluated under the following standard reaction conditions:

Reaction Temperature=60° C.

Ethylene Pressure=23 bar

Al/Ti molar ratio=2

Reaction Time=1 hour

Agitator Speed=600 rpm

n-Hexane amount=50 ml

The titanium concentration in commercial Alphabutol™ system is about 7.71 wt. %.

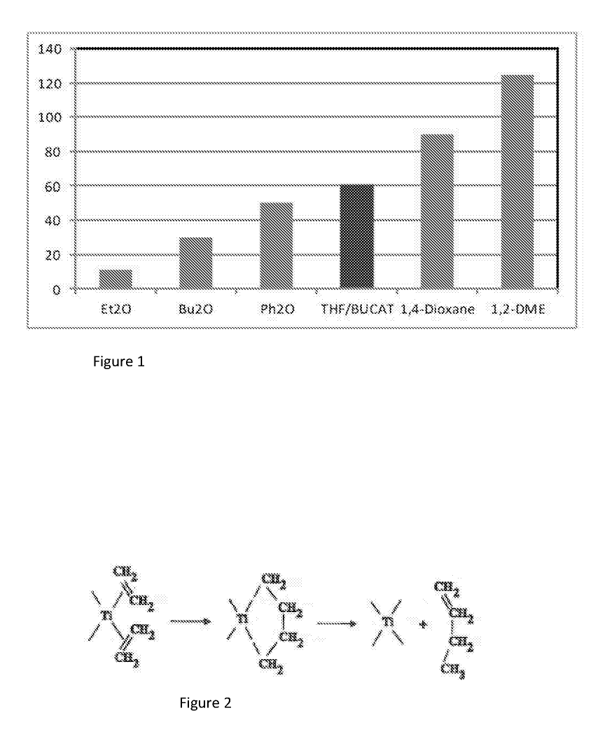

A reduction reaction was observed immediately, e.g., the ethylene consumption was about 40 g after 10 minutes, and was about 50 g after 15 minutes. The induction period was about one minute, which is much shorter than the induction period of a catalytic solution that does not include a diether but rather includes a THF (usually about 8-12 minutes). The total ethylene consumption after 1 hour was about 86.4 g, as shown in FIG. 1.

Separately, 2.78 ml of TNBT was mixed with 3.17 ml of 1,2-DME. About 2.2 ml of 1M TEAL dissolved in 50 ml of n-hexane was added to the mixture of TNBT/1,2-DME to obtain a catalytic solution used for producing 1-butene as described in Example 1 above. The total ethylene consumption after 1 hour was about 125.4 g, as shown in FIG. 1. The induction period was about one minute. There was no fouling observed.

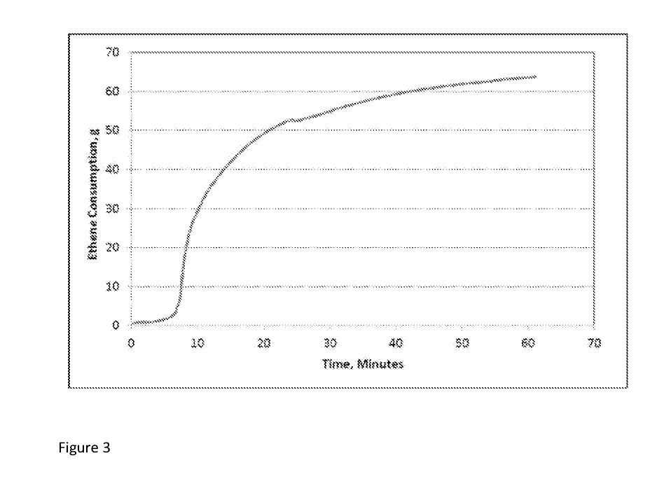

For comparison, a 0.5 ml of a catalytic solution including 45 vol. % THF and 55 vol % of TNBT was dissolved in 50 ml n-hexane prepared in a glove box under a nitrogen atmosphere. Prior to use, 1.9 ml of 1M TEAL solution was added. A lengthy induction period of about 8 minutes was observed. The total ethylene consumption after 1 hour was about 65.1 g, as shown in FIG. 1 and FIG. 3.

The catalytic activity of catalytic solutions including TNBT and one of the following monoethers: diethylether, dibutylether, and diphenylether were also tested for comparison. The total ethylene consumptions after 1 hour for diethylether, dibutylether, and diphenylether were about 15 g, about 30 g, and about 50 g, respectively.

### Example 2

Mixtures of TNBT, 1,4-dioxane and THF were prepared. These mixtures were subsequently mixed with about 2.3 ml of 1M TEAL dissolved in 50 ml of n-hexane to obtain catalytic solutions used for producing 1-butene as described in Example 1 above. The total ethylene consumptions after 1 hour of these mixtures are shown in Table 1.

TABLE 1

| | | | | Total ethylene | |
|---|---|---|---|---|---|
| | | 1,4-dioxane | | consumption | Fouling |
| | TNBT (wt %) | (wt %) [volume] | THF (wt %) | after 1 hour (g) | observed? |
| 1 | 55 [2.78 ml] | 35 [1.06 ml] | 10 [0.45 ml] | 89.3 | No |
| 2 | 55 [2.78 ml] | 40 [3.06 ml] | 5 [0.22 ml] | 65 | No |
| 3 | 55 [2.78 ml] | 22.5 [1.16 ml] | 22.5 [1.0 ml] | 89.9 | No |
| 4 | 55 [2.78 ml] | 10 [0.515 ml] | 35 [1.54 ml] | 5.4 | No |
| 5 | 55 [2.78 ml] | 5 [0.258 ml] | 40 [1.28 ml] | 79.9 | No |

The invention is further illustrated by the following embodiments.

### Embodiment 1

A catalyst composition, comprising an organic titanium compound, an organic aluminum compound, and a cyclic diether, a linear diether, or a combination comprising at least one of the foregoing.

### Embodiment 2

The catalyst composition of Embodiment 1, wherein the organic titanium compound is a titanate of the formula Ti(OR)₄ wherein each R is the same or different and is a C₁₋₁₂ alkyl group, preferably wherein the organic titanium compound is titanium tetra-n-butoxide.

### Embodiment 3

The catalyst composition of Embodiment 1 or 2, wherein the organic aluminum compound is of the formula AlₙR₃ₙ, wherein n is 1 or 2 and each R is the same or different and is a C₁₋₁₂ alkyl group, preferably wherein the organic aluminum compound is triethylaluminum.

### Embodiment 4

The catalyst composition of any one or more Embodiments 1 to 3, wherein the linear diether is a symmetrical or unsymmetrical diether of the formula (CₙH₂ₙ₊₁)O(CₙH₂ₙ)O(CₙH₂ₙ₊₁) wherein for each moiety n is the same or different, and is 1 to 4, preferably 1 to 2.

### Embodiment 5

The catalyst composition of Embodiment 4, wherein the linear diether is 1,2-dimethoxyethane.

### Embodiment 6

The catalyst composition of Embodiment 4 or Embodiment 5, wherein the organic titanium compound is titanium tetra-n-butoxide, and the organic aluminum compound is triethylaluminum.

### Embodiment 7

The catalyst composition of any one or more Embodiments 1 to 3, wherein the cyclic diether is a 1,4-dioxane optionally substituted with 1, 2, or 3 halogen, C₁-C₆ alkyl, C₆₋₁₀ aryl, or C₂-C₆ alkenyl substituents.

### Embodiment 8

The catalyst composition of Embodiment 7, wherein the cyclic diether is 1,4-dioxane.

### Embodiment 9

The catalyst composition of Embodiment 7 or 8, wherein the organic titanium compound is titanium tetra-n-butoxide, and the organic aluminum compound is triethylaluminum, and

### Embodiment 10

The catalyst composition of any one or more Embodiments 1 to 8, wherein catalyst composition further comprises tetrahydrofuran.

### Embodiment 11

A process for converting ethylene to 1-butene, the process comprising: contacting ethylene with the catalyst composition of any one or more of Embodiments 1 to under conditions effective to form the 1-butene.

### Embodiment 12

The process of any one or more of Embodiment 11, wherein the conditions comprise a pressure of about 1 to about 120 bar, preferably about 5 to about 50 bar, and a temperature of about 30 to about 150° C., preferably about 40 to about 80° C.

### Embodiment 13

The process of Embodiment 11, wherein the total ethylene consumption after about 1 hour is about 125 g when the process is performed at a temperature of from about 55° C. to about 60° C. and a pressure of from about 20 bars to about 25 bars.

### Embodiment 14

A process for the preparation of a downstream product, the process comprising: reacting the 1-butene prepared according to any one or more of Embodiments 11 to 13, to provide the downstream product, preferably wherein the downstream product is a homopolymer or copolymer comprising units derived from the α-olefin.

### Embodiment 15

The process of Embodiment 14, further comprising shaping the downstream product to provide an article.

All publications, patents, and patent applications cited herein are hereby expressly incorporated by reference for all purposes to the same extent as if each was so individually denoted.

The term “about” or “substantially” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. The singular forms “a,” “an,” and “the” include plural referents unless the context clearly dictates otherwise. “Or” means “and/or.” Unless defined otherwise, technical and scientific terms used herein have the same meaning as is commonly understood by one of skill in the art to which this invention belongs.

Although the presently disclosed subject matter and its advantages have been described in detail, it should be understood that various changes, substitutions, and alterations can be made herein without departing from the spirit and scope of the presently disclosed subject matter as defined by the appended claims. Moreover, the scope of the presently disclosed subject matter is not intended to be limited to the particular embodiments described in the specification. Accordingly, the appended claims are intended to include within their scope such modifications.

1. A catalyst composition, comprising an organic titanium compound, an organic aluminum compound, and a cyclic diether, a linear diether, or a combination comprising at least one of the foregoing.

2. The catalyst composition of claim 1, wherein the organic titanium compound is a titanate of the formula Ti(OR)₄ wherein each R is the same or different and is a C₁₋₁₂ alkyl group.

3. The catalyst composition of claim 2, wherein the organic aluminum compound is of the formula AlₙR₃ₙ, wherein n is 1 or 2 and each R is the same or different and is a C₁₋₁₂ alkyl group.

4. The catalyst composition of claim 3, wherein the linear diether is a symmetrical or unsymmetrical diether of the formula (CₙH₂ₙ₊₁)O(CₙH₂ₙ)O(CₙH₂ₙ₊₁) wherein for each moiety n is the same or different, and is 1 to 4.

5. The catalyst composition of claim 4, wherein the linear diether is 1,2-dimethoxyethane.

6. The catalyst composition of claim 5, wherein the organic titanium compound is titanium tetra-n-butoxide, and the organic aluminum compound is triethylaluminum.

7. The catalyst composition claim 3, wherein the cyclic diether is a 1,4-dioxane optionally substituted with 1, 2, or 3 halogen, C₁-C₆ alkyl, C₆₋₁₀ aryl, or C₂-C₆ alkenyl substituents.

8. The catalyst composition of claim 7, wherein the cyclic diether is 1,4-dioxane.

9. The catalyst composition of claim 8, wherein the organic titanium compound is titanium tetra-n-butoxide, and the organic aluminum compound is triethylaluminum.

10. The catalyst composition of claim 1, wherein the catalyst composition further comprises tetrahydrofuran.

11. A process for converting ethylene to 1-butene, the process comprising:
contacting ethylene with the catalyst composition of claim 1 under conditions effective to form the 1-butene.

12. The process of claim 11, wherein the conditions comprise
a pressure of about 1 to about 120 bar, and
a temperature of about 30 to about 150° C.

13. The process of claim 11, wherein the total ethylene consumption after about 1 hour is about 125 g when the process is performed at a temperature of from about 55° C. to about 60° C. and a pressure of from about 20 bar to about 25 bar.

14. A process for the preparation of a downstream product, the process comprising:
reacting the 1-butene prepared according to the process of claim 11, to provide the downstream product, wherein the downstream product is a homopolymer or copolymer comprising units derived from the 1-butene.

15. The process of claim 14, further comprising shaping the downstream product to provide an article.

16. The catalyst composition of claim 2, wherein the organic titanium compound comprises titanium tetra-n-butoxide.

17. The catalyst composition of claim 3, wherein the organic aluminum compound comprises triethylaluminum.

18. The catalyst composition of claim 4, wherein the linear diether is a symmetrical or unsymmetrical diether of the formula (CₙH₂ₙ₊₁)O(CₙH₂ₙ)O(CₙH₂ₙ₊₁), wherein for each moiety n is the same or different, and is 1 to 2.

19. The catalyst composition of claim 1, wherein the organic titanium compound comprises titanium tetra-n-butoxide, the organic aluminum compound comprises triethylaluminum, the linear diether is the linear diether is 1,2-dimethoxyethane, and the cyclic diether is 1,4-dioxane.

20. The process of claim 12, wherein the conditions further comprise a pressure of about 5 bar to about 50 bar and a temperature of about 40° C. to about 80° C.
